(19) United States
(12) Patent Application Publication
(10) Pub. No.: US 2019/0070002 A1
(43) Pub. Date: Mar. 7, 2019

(54) MEDICAL DEVICE DELIVERY SYSTEM WITH FLEXIBLE COUPLER

(71) Applicant: BOSTON SCIENTIFIC SCIMED, INC., Maple Grove, MN (US)

(72) Inventors: Mimi Trinh Fitterer, Belmont, CA (US); Takashi H. Ino, San Jose, CA (US); Ian M. Mahaffey, Westley, CA (US); Katherine Sapozhnikov, Campbell, CA (US); Michael P. Calomeni, San Jose, CA (US)

(73) Assignee: BOSTON SCIENTIFIC SCIMED, INC., MAPLE GROVE, MN (US)

(21) Appl. No.: 16/116,109

(22) Filed: Aug. 29, 2018

Related U.S. Application Data
(60) Provisional application No. 62/554,234, filed on Sep. 5, 2017.

(51) Int. Cl. A61F 2/24; A61F 2/966

(52) U.S. Cl. CPC A61F 2/2436; A61F 2002/9517; A61F 2002/9665; A61F 2/966

(57) ABSTRACT

A medical device delivery system includes an outer shaft having an outer shaft lumen extending therethrough and an inner shaft that is slidingly disposable within the outer shaft lumen. A coupler assembly is secured to a distal end region of the inner shaft and includes an elongate tubular member including a proximal region and a distal region, the proximal region of the elongate tubular member secured to the distal end region of the inner shaft, a plurality of fingers extending distally from the distal region of the elongate tubular member that are adapted to releasably engage an implantable medical device and a plurality of sheathing aids extending distally from the distal region of the elongate tubular member that are adapted to guide the medical device back into the outer shaft lumen.

### CROSS-REFERENCE TO RELATED APPLICATIONS

This application claims the benefit of priority under 35 U.S.C. § 119 to U.S. Provisional Application Ser. No. 62/554,234, filed Sep. 5, 2017, the entirety of which is incorporated herein by reference.

### TECHNICAL FIELD

The present disclosure pertains generally to valve delivery devices and more particularly to valve delivery devices that facilitate alignment of the valve to the native annulus.

### BACKGROUND

Medical devices typically used for cardiovascular system treatments may involve complex and invasive therapies resulting in significant discomfort, pain, and long recovery times for patients. Recently, less invasive, percutaneous treatments have been developed. There is an ongoing need for improved, less invasive cardiovascular treatments.

### SUMMARY

The disclosure provides design, material, and manufacturing method alternatives for valve delivery devices, particularly valve delivery devices that facilitate coaxial alignment of the valve with the native annulus. An example of the disclosure is a medical device delivery system that includes an outer shaft having an outer shaft lumen extending therethrough and an inner shaft that is slidingly disposable within the outer shaft lumen and that includes a distal end region. A coupler is secured to the distal end region and includes an elongate tubular member having a proximal region and a distal region, the proximal region of the elongate tubular member secured to the distal end region of the inner shaft. A plurality of fingers extend distally from the distal region of the elongate tubular member and are adapted to releasably engage an implantable medical device. A plurality of sheathing aids extend distally from the distal region of the elongate tubular member and are adapted to guide the medical device back into the outer shaft lumen.

Alternatively or additionally to any embodiment above, the elongate tubular member may further include one or more windows cut into the proximal region of the elongate tubular member in order to facilitate securement of the elongate tubular member to the distal end region of the inner shaft.

Alternatively or additionally to any embodiment above, the coupler may be laser cut from a single piece of metal.

Alternatively or additionally to any embodiment above, the plurality of fingers may be part of the elongate tubular member.

Alternatively or additionally to any embodiment above, the plurality of sheathing aids may be part of the elongate tubular member.

Alternatively or additionally to any embodiment above, at least some of the plurality of fingers and/or at least some of the plurality of sheathing aids may be welded or soldered to the distal region of the elongate tubular member.

Alternatively or additionally to any embodiment above, the elongate tubular member may be laser cut to provide the elongate tubular member with a spine running along one side of the elongate tubular member and a plurality of slots extending radially from the spine in order to provide the elongate tubular member with a preferential bending direction.

Alternatively or additionally to any embodiment above, the elongate tubular member may preferentially bend in a preferential bending direction away from the spine, the plurality of slots closing up to enable the elongate tubular member to bend in the preferential bending direction.

Alternatively or additionally to any embodiment above, the coupler assembly may further include a polymeric liner disposed about the elongate tubular member.

Alternatively or additionally to any embodiment above, at least some of the plurality of fingers may include a serrated section adapted to secure a guide member to the finger.

Alternatively or additionally to any embodiment above, the elongate tubular member may include a polymeric tubular member.

Alternatively or additionally to any embodiment above, the implantable medical device may include an implantable heart valve.

Another example of the disclosure is a medical device delivery system that includes an outer shaft having an outer shaft lumen extending therethrough and an inner shaft that is slidingly disposable within the outer shaft lumen and that includes an inner shaft lumen extending therethrough, the inner shaft including a distal end region. A laser cut coupler is secured to the distal end region and includes an elongate tubular member and a plurality of fingers extending distally from the elongate tubular member, the plurality of fingers adapted to engage an implantable medical device. The elongate tubular member is laser cut to provide the elongate tubular member with a spine running along one side of the elongate tubular member and a plurality of slots extending radially from the spine in order to provide the elongate tubular member with a preferential bending direction in which the elongate tubular member bends away from the spine.

Alternatively or additionally to any embodiment above, the plurality of slots may close up to enable the elongate tubular member to bend in the preferential bending direction.

Alternatively or additionally to any embodiment above, the medical device delivery system may further include a plurality of sheathing aids that extend distally from the elongate tubular member and that are adapted to guide the medical device back into the outer shaft lumen.

Alternatively or additionally to any embodiment above, the medical device delivery system may further include an actuation member extending through the inner shaft lumen and operably coupled to the elongate tubular member such that actuation of the actuation member causes the elongate tubular member to bend in the preferential bending direction.

Alternatively or additionally to any embodiment above, the elongate tubular member and the plurality of fingers may be laser cut from a single piece of metal.

Alternatively or additionally to any embodiment above, the outer shaft may have a curved distal end.

Alternatively or additionally to any embodiment above, the inner shaft may have a curved distal end.

Another example of the disclosure is a medical device delivery system that includes an outer shaft having an outer shaft lumen extending therethrough, an inner shaft that is slidingly disposable within the outer shaft lumen and a coupler that extends distally from the inner shaft. The coupler includes an elongate tubular member having a proximal region and a distal region, the proximal region of the elongate tubular member secured to the distal end region of the inner shaft. A plurality of fingers extend distally from the distal region of the elongate tubular member and are adapted to releasably engage an implantable heart valve. A plurality of sheathing aids extend distally from the distal region of the elongate tubular member and are adapted to guide the implantable heart valve back into the outer shaft lumen.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is side view of an example medical device system;

FIG. 2 is a cross-sectional side view of an example outer sheath;

FIG. 3 is a transverse cross-sectional view taken through line 3-3 in FIG. 2;

FIG. 4 is a side view of an example inner catheter;

FIG. 5 is a cross-sectional view taken through line 5-5 in FIG. 4;

FIG. 6 is a cross-sectional view taken through line 6-6 in FIG. 4;

FIG. 7 is a perspective view of a portion of an example implant associated with the example medical device system;

FIG. 8 through FIG. 11 are perspective views that illustrate an example mechanism for locking an implant;

FIG. 12 is a perspective view of an example inner catheter in a straight configuration;

FIG. 13 is an end view of the inner catheter of FIG. 12;

FIG. 14 is a perspective view of the inner catheter of FIG. 12, shown in a deflected configuration;

FIG. 15 is a perspective view of an example coupler in a first orientation;

FIG. 16 is a perspective view of the coupler of FIG. 16, shown rotated 180 degrees; and

FIG. 17 is a perspective view of an example inner catheter.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Diseases and/or medical conditions that impact the cardiovascular system are prevalent in the United States and throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve can have a serious effect on a human and could lead to serious health condition and/or death if not dealt with. Treatment of defective heart valves poses other challenges in that the treatment often requires the repair or outright replacement of the defective valve. Such therapies may be highly invasive to the patient. Disclosed herein are medical devices that may be used for delivering a medical device to a portion of the cardiovascular system in order to diagnose, treat, and/or repair the system. At least some of the medical devices disclosed herein may be used to deliver and implant a replacement heart valve (e.g., a replacement aortic valve). In addition, the devices disclosed herein may deliver the replacement heart valve percutaneously and, thus, may be much less invasive to the patient. The devices disclosed herein may also provide a number of additional desirable features and benefits as described in more detail below.

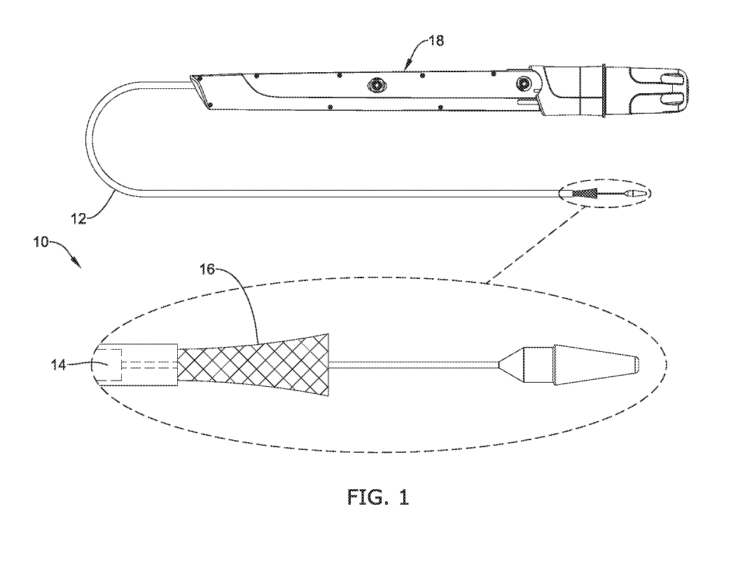

FIG. 1 is a side view of an example medical device system 10. It should be noted that some features of the system 10 are either not shown, or are shown schematically, in FIG. 1 for simplicity. Additional details regarding some of the components of the system 10 are provided in other figures in greater detail. The system 10 may be used to deliver and/or deploy a variety of medical devices to a number of locations within the anatomy. In at least some cases, the system 10 is a replacement heart valve delivery system (e.g., a replacement aortic valve delivery system) that can be used for percutaneous delivery of a replacement heart valve. This, however, is not intended to be limiting as the system 10 may also be used for other interventions including mitral valve replacement, valve repair, valvuloplasty, and the like, or other similar interventions.

The system 10 may generally be described as a catheter system that includes an outer sheath or catheter 12 and an inner catheter or tube 14 (a portion of which is shown in FIG. 1 in phantom line) extending at least partially through the outer sheath 12. A medical device implant 16 may be coupled to the inner catheter 14 and disposed within the outer sheath 12 during delivery of the implant 16. A handle 18 may be disposed at the proximal end of the outer sheath 12 and the inner catheter 14. In general, the handle 18 may be configured to manipulate the position of the outer sheath 12 relative to the inner catheter 14 as well as aid in the deployment of the implant 16.

In use, the system 10 may be advanced percutaneously through the vasculature to a position adjacent to an area of interest. For example, the system 10 may be advanced through the vasculature to a position adjacent to a defective aortic valve. During delivery, the implant 16 may be generally disposed in an elongated and low profile “delivery” configuration within the outer sheath 12. Once positioned, the outer sheath 12 may be retracted to expose the implant 16. The implant 16 may be actuated in order to expand implant into a generally shortened and larger profile “deployed” configuration suitable for implantation within the anatomy. When the implant 16 is suitably deployed within the anatomy, the system 10 can be removed from the vasculature, leaving the implant 16 in place to function as, for example, a suitable replacement for the native aortic valve. In at least some interventions, the implant 16 may be deployed within the native valve (e.g., the native valve is left in place and not excised). Alternatively, the native valve may be removed and implant 16 may be deployed in its place as a replacement.

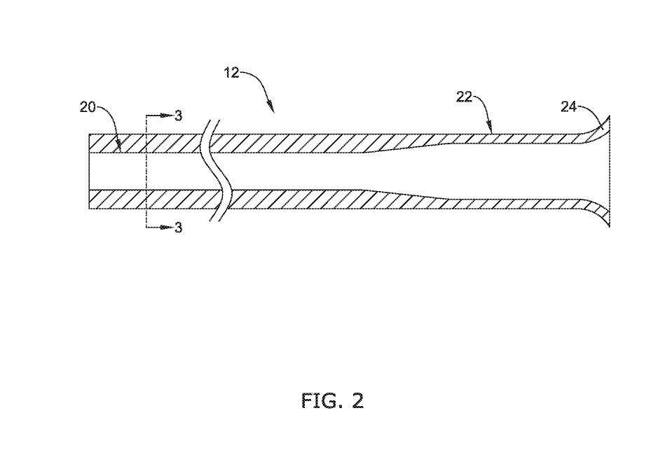

FIG. 2 through FIG. 11 illustrate some of the components of the system 10. For example, FIG. 2 is a cross-sectional side view of the outer sheath 12. Here it can be seen that the outer sheath 12 has a proximal portion 20 and a distal portion 22. The distal portion 22 may have a slightly enlarged or flared inner diameter, which may provide additional space for holding the implant 16 therein. For example, the inner diameter of the outer sheath 12 along the proximal portion 20 may be in the range of about 0.254 to 1.27 cm (0.10 to 0.50 inches), or about 0.508 to 1.016 cm (0.20 to 0.40 inches), or about 0.508 to 0.762 cm (0.20 to 0.30 inches), or about 0.56388±0.0508 cm (0.222±0.002 inches). The inner diameter of the outer sheath 12 along the distal portion 22 may be in the range of about 0.254 to 1.27 cm (0.10 to 0.50 inches), or about 0.508 to 1.016 cm (0.20 to 0.40 inches), or about 0.508 to 0.762 cm (0.20 to 0.30 inches), or about 0.579 to 0.5842 cm (0.228 to 0.230 inches). At the distal end of the distal portion 22 may be a distal tip 24, which may be flared or otherwise have a funnel-like shape. The funnel-like shape increases the outer diameter (and inner diameter) of the outer sheath 12 at the distal tip 24 and may aid in the sheathing and/or re-sheathing of the implant 16 into the outer sheath 12. Other than at the distal tip 24, the outer sheath 12 may have a generally constant outer diameter. For example, the outer sheath 12 may have an outer diameter in the range of about 0.254 to 1.27 cm (0.10 to 0.50 inches), or about 0.508 to 1.016 cm (0.20 to 0.40 inches), or about 0.508 to 0.762 cm (0.20 to 0.30 inches), or about 0.6858 cm (0.270 inches). These are just examples. Other embodiments are contemplated that have differing dimensions (including those appropriate for differently sized patients including children) and/or arrangements for the outer diameter and/or inner diameter of the outer sheath 12. These contemplated embodiments include outer sheaths with flared or otherwise variable outer diameters, embodiments with constant inner diameters, combinations thereof, and the like. The outer sheath 12 may also have a length that is appropriate for reaching the intended area of interest within the anatomy. For example, the outer sheath 12 may have a length in the range of about 30 to 200 cm, or about 60 to 150 cm, or about 100 to 120 cm, or about 108±0.20 cm. The outer sheath 12 may also be curved. For example, a distal section of the outer sheath 12 may be curved. In one example, the radius of the curve (measured from the center of the outer sheath 12) may be in the range of about 2 to 6 cm (20 to 60 mm), or about 3 to 4 cm (30 to 40 mm), or about 3.675 cm (36.75 mm). Again, these dimensions are examples and are not intended to be limiting.

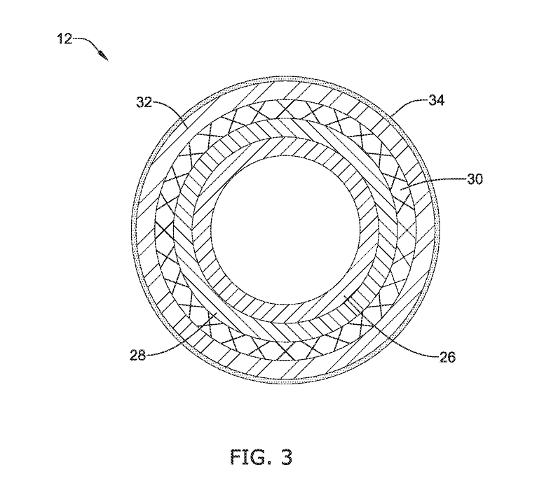

The outer sheath 12 may be formed from a singular monolithic tube or unitary member. Alternatively, the outer sheath 12 may include a plurality of layers or portions. One or more of these layers may include a reinforcing structure such as a braid, coil, mesh, combinations thereof, or the like. FIG. 3 illustrates one example of a multilayer structure for the outer sheath 12. For example, the outer sheath 12 may include an inner liner or layer 26. An intermediate or tier layer 28 may be disposed on the inner liner 26. A reinforcement 30 may be disposed on the intermediate layer 28. A topcoat or outer layer 32 may be disposed on the reinforcement 30. Finally, an outer coating 34 (e.g., a lubricious coating, a hydrophilic coating, a hydrophobic coating, etc.) may be disposed along portions or all of the topcoat 32. These are just examples. Several alternative structural configurations are contemplated for the outer sheath 12 including embodiments including two or more layers that may be different from those shown in FIG. 3, embodiments without a reinforcement, and the like, or other suitable configurations.

The dimensions and materials utilized for the various layers of the outer sheath 12 may also vary. For example, the inner liner 26 may include a polymeric material such as fluorinated ethylene propylene (FEP) and may have a thickness in the range of about 0.00254 to 0.0127 cm (0.001 to 0.005 inches) or about 0.00762±0.00254 (0.003±0.001 inches), the intermediate layer 28 may include a polymer material such as polyether block amide (e.g., PEBAX 6333) and may have a thickness in the range of about 0.00254 to 0.0127 cm (0.001 to 0.005 inches) or about 0.00508±0.00254 (0.002±0.001 inches), the outer coating 34 may include a polymer material such as polyether block amide (e.g., PEBAX 7233) and may have a thickness in the range of about 0.00254 to 0.0254 cm (0.001 to 0.01 inches). In some embodiments, the outer coating 34 may vary in thickness. For example, along the proximal portion 20 the outer coating 34 may have greater thickness, such as about 0.0127 to about 0.0508 cm or about 0.02159 cm (0.005 to 0.02 inches or about 0.0085 inches), than along the distal portion 22 and/or the distal tip 24, which may be about 0.0127 to about 0.0508 cm or about 0.01651 cm (e.g., about 0.005 to 0.02 inches or about 0.0065 inches). These are just examples as other suitable materials may be used.

The form of the distal tip 24 may also vary. For example, in at least some embodiments, the inner liner 26 (i.e., a 2.5 mm section thereof) may be extended up and around the distal end of the outer sheath 12 (e.g., around the reinforcement 30 and the topcoat 32). A ring member (not shown) made from a suitable material such as a 55 D polyether block amide (e.g., 55 D PEBAX) may be disposed over the inner liner 26 and heat bonded to form the distal tip 24. This may form the funnel-like shape of the distal tip 24.

The reinforcement 30 may also vary in form. In at least some embodiments, the reinforcement 30 may take the form of a braid, coil, mesh, or the like. For example, in some embodiments, the reinforcement 30 may include a metallic braid (e.g., stainless steel). In some of these embodiments, the reinforcement 30 may also include additional structures such as one or more longitudinally-extending strands. For example, the reinforcement 30 may include a pair of longitudinally-extending aramid and/or para aramid strands (for example, KEVLAR®) disposed on opposite sides of the braid. These strands may or may not be woven into portions or all of the braid.

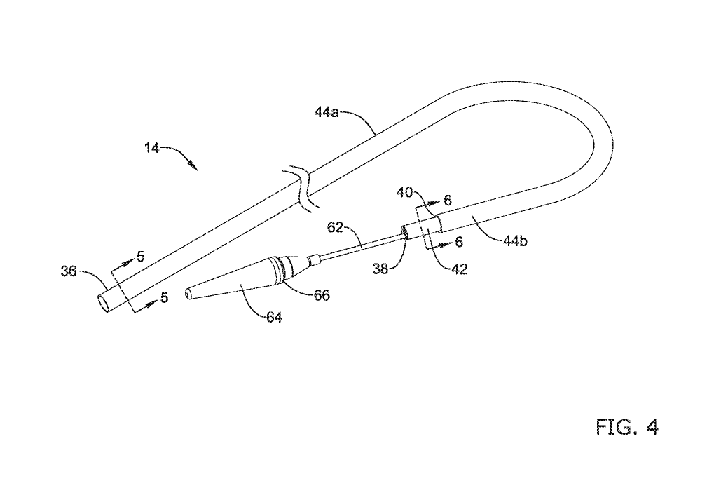

FIG. 4 is a side view of the inner catheter 14. A distal end region of the inner catheter 14 may include a step 40 in outer diameter that defines a decreased outer diameter section 42. For example, the decreased outer diameter section 42 may have an outer diameter in the range of about 0.127 to 0.635 cm (0.05 to 0.25 inches), or about 0.254 to 0.508 cm (0.10 to 0.20 inches), or about 0.38608±0.00762 (0.152±0.003 inches) as opposed to the remainder of the inner catheter 14 where the outer diameter may be in the range of about 0.127 to 0.762 cm (0.05 to 0.30 inches), or about 0.254 to 0.635 cm (0.10 to 0.25 inches), or about 0.508±0.0254 cm (0.20±0.01 inches). The decreased outer diameter section 42 may define a region where other components of the system 10 may be attached. Some additional details regarding these components can be found herein.

In general, the inner catheter 14 may take the form of an extruded polymer tube. Other forms are also contemplated including other polymer tubes, metallic tubes, reinforced tubes, or the like including other suitable materials such as those disclosed herein. In some embodiments, the inner catheter 14 is a singular monolithic or unitary member. In other embodiments, the inner catheter 14 may include a plurality of portions or segments that are coupled together. The total length of the inner catheter may be in the range of about 60 to 150 cm, or about 80 to 120 cm, or about 100 to 115 cm, or about 112±0.02 cm. Just like the outer sheath 12, the inner catheter 14 may also be curved, for example adjacent to the distal end thereof. In some embodiments, the inner catheter 14 may have one or more sections with a differing hardness/stiffness (e.g., differing shore durometer). For example, the inner catheter may have a proximal region 44a and an intermediate region 44b. The proximal region 44a may include a generally stiff polymeric material such as a 72 D polyether block amide (e.g., 72 D PEBAX) and may have a length in the range of about 60 to 150 cm, or about 80 to 120 cm, or about 100 to 115 cm, or about 109.5±0.02 cm. The intermediate region 44b may include a 40 D polyether block amide (e.g., 40 D PEBAX) and may have a length in the range of about 5 to 25 mm, or about 10 to 20 mm, or about 15±0.01 mm. The decreased outer diameter section 42 may also differ from regions 44a/44b and, in some embodiments, may include a 72 D polyether block amide (e.g., 72 D PEBAX) and may have a length in the range of about 0.5 to 2 cm (5 to 20 mm), or about 0.8 to 1.5 cm (8 to 15 mm), or about 1±0.001 cm (10±0.01 mm). These are just examples.

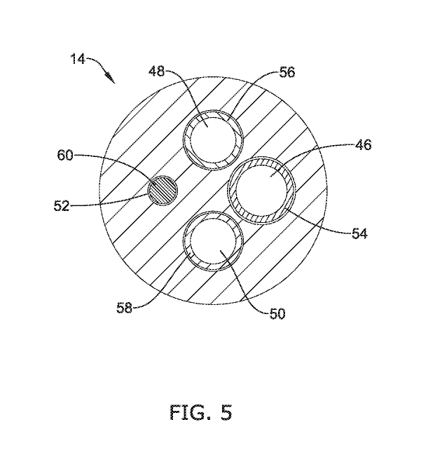

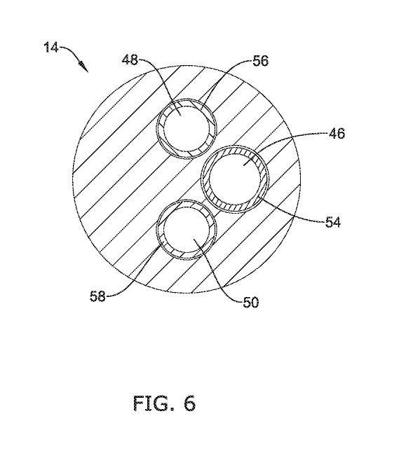

The inner catheter 14 may include one or more lumens. For example, FIG. 5, a cross-sectional view of the inner catheter 14 adjacent to a proximal end portion 36 taken at line 5-5 in FIG. 4, illustrates that the inner catheter 14 may include a first lumen 46, a second lumen 48, a third lumen 50, and a fourth lumen 52. In general, the lumens 46/48/50/52 extend along the entire length of the inner catheter 14. Other embodiments are contemplated, however, where one or more of the lumens 46/48/50/52 extend along only a portion of the length of the inner catheter 14. For example, the fourth lumen 52 may stop just short of the distal end of the inner catheter 14 and/or be filled in at its distal end to effectively end the fourth lumen 52 proximal of the distal end of the inner catheter 14. For example, as illustrated in FIG. 6, which is a cross-sectional view of the inner catheter 14 taken at line 6-6 in FIG. 4, the fourth lumen 52 is absent.

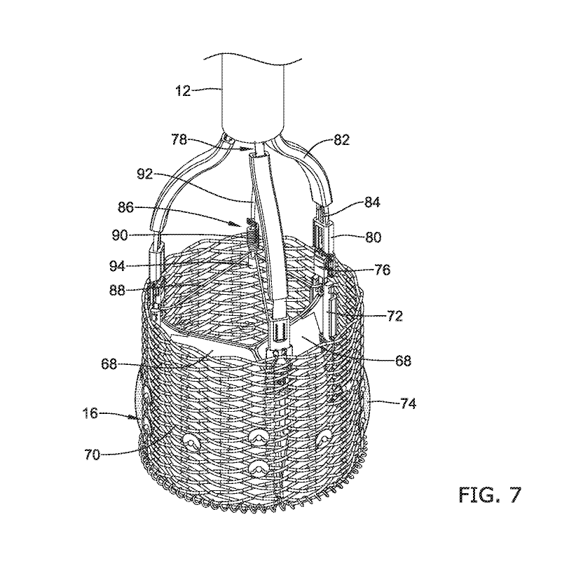

Disposed within the first lumen 46 may be push-pull rods 84 (not shown in FIG. 5, seen in other figures including FIG. 7), which are used to expand and/or elongate the implant 16 as explained in more detail herein. In at least some embodiments, the first lumen 46 may be lined with a low friction liner 54 (e.g., a FEP liner). Disposed within the second lumen 48 may be a pin release mandrel 92 (not shown in FIG. 5, seen in other figures including FIG. 7), which is explained in more detail herein. In at least some embodiments, the second lumen 48 may be lined with a hypotube liner 56. The third lumen 50 may be a guidewire lumen and this lumen may also be lined with a hypotube liner 58.

The fourth lumen 52 may be used to house a non-stretch wire 60. The form of the non-stretch wire 60 may vary. In some embodiments, the non-stretch wire 60 may take the form of a stainless steel braid. The non-stretch wire 60 may optionally include a pair of longitudinally-extending aramid and/or para aramid strands (for example, KEVLAR®) disposed on opposite sides of the braid. In general, rather than being “disposed within” the fourth lumen 52, the non-stretch wire 60 may be embedded within the fourth lumen 52. In addition, the non-stretch wire 60 may extend to a position adjacent to the distal end portion 38 but not fully to the distal end of the inner catheter 14 as illustrated in FIG. 6 by the absence of the fourth lumen 52 adjacent to the distal end of the inner catheter 14. For example, a short distal segment of the fourth lumen 52 may be filled in with polymer material adjacent to the distal end of the inner catheter 14.

Returning to FIG. 4, the inner catheter 14 may also include a guidewire extension tube 62 that extends distally from the distal end portion 38. A nose cone 64 is attached to the guidewire extension tube 62. The nose cone 64 generally is designed to have an atraumatic shape. The nose cone 64 may also include a ridge or ledge 66 that is configured to abut the distal tip 24 of the outer sheath 12 during delivery of the implant 16.

FIG. 7 illustrates some of the additional components of the system 10 and the implant 16. For example, here it can be seen that the implant 16 includes a plurality of valve leaflets 68 (e.g., bovine pericardial) which are secured to a cylindrical braid 70 at a post or commissure post 72, for example at the commissure portions of the leaflets 68. In this example, the implant 16 includes three leaflets 68 secured to the braid 70 with three posts 72. The leaflets 68 may also be secured to the base or “distal end” of the braid 70. The posts 72, in turn, may be secured to the braid 70 (e.g., along the interior of the braid 70) with sutures or other suitable mechanisms. Positioned adjacent to (e.g., longitudinally spaced from and aligned with) the posts 72 are a plurality of buckles 76, which may also be sutured to the braid 70 (e.g., along the interior of the braid 70). In this example, one buckle 76 is attached to the braid 70 adjacent to each of the three posts 72. Accordingly, the braid 70 has a total of three buckles 76 and three posts 72 attached thereto. Other embodiments are contemplated where fewer or more buckles 76 and posts 72 may be utilized. A seal 74 (shown in cross-section) may be disposed about the braid 70 and, as the name suggests, may help to seal the implant 16 within a target implant site or area of interest.

Attachment between the implant 16 and the inner catheter 14 (and/or outer sheath 12) may be effected through the use of a three finger coupler 78. It will be appreciated that the coupler 78 is merely an example, as other couplers may include additional components not shown with the coupler 78. The coupler 78 may generally include a cylindrical base (not shown) that is attached to the inner catheter 14 (e.g., disposed about and attached to the reduced outer diameter section 42). Projecting distally from the base are three fingers that are each configured to engage with the implant 16 at the posts 72 and the buckles 76. A collar 80 may further assist in holding together these structures. A guide 82 may be disposed over each of the fingers and may serve to keep the fingers of the coupler 78 associated with push-pull rods 84 extending adjacent to the coupler 78. Finally, a pin release assembly 86 may be a linking structure that keeps the posts 72, the buckles 76, and the push-pull rods 84 associated with one another. The pin release assembly 86 includes a plurality of individual pins 88 that may be joined together via a coiled connection 90 and held to a pin release mandrel 92 with a ferrule 94.

During delivery, the implant 16 is secured at the distal end of the inner catheter 14 by virtue of the association of the fingers of the coupler 78 being coupled with a projecting proximal end of the buckles 76 (and being held in place with the collar 80 disposed over the connection) and by virtue of the pins 88 securing together the push-pull rods 84 and the posts 72. When the implant 16 is advanced within the anatomy to the desired location, the outer sheath 12 may be withdrawn (e.g., moved proximally relative to the inner catheter 14) to expose the implant 16. Then, the push-pull rods 84 can be used to expand and “lock” the implant 16 in the expanded or deployed configuration by proximally retracting the push-pull rods 84 to pull the posts 72 into engagement with the buckles 76. Finally, the pins 88 can be removed, thereby uncoupling the push-pull rods 84 from the posts 72, which allows the implant 16 to be released from the system 10 and deployed in the anatomy.

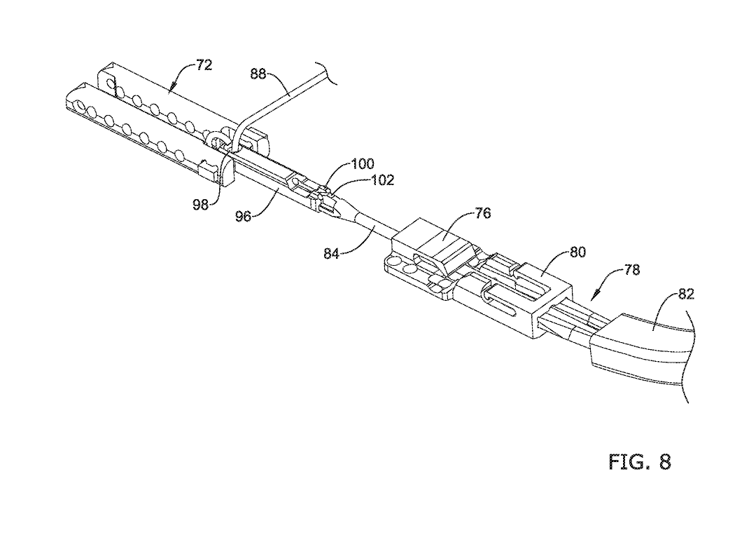

FIG. 8 through FIG. 11 illustrate the locking system utilized with the system 10. For simplicity purposes, only one of the three fingers of the coupler 78, only one of the three push-pull rods 84, and only one of the posts 72 of the example system 10 are shown (and the implant 16 is not shown). As seen in FIG. 8, the push-pull rod 84 extends through the guide 82 adjacent to the fingers of the coupler 78, through the collar 80, through the buckle 76, and into a hollow t-shaped bar portion 96 of the post 72. The distal end of the push-pull rod 84 may include an opening or aperture (not shown) that can be aligned with an opening 98 of the t-shaped bar portion 96. When so aligned, the pin 88 can be looped through the opening 98 and the opening of the push-pull rod 84. This secures the push-pull rod 84 to the post 72 and forms a configuration of these structures that can be utilized during delivery of the implant 16. As can be appreciated, the proximal end of the post 72 and the distal end of the buckle 76 are longitudinally separated and, accordingly, the implant 16 is in an elongated and generally low-profile configuration suitable for delivery.

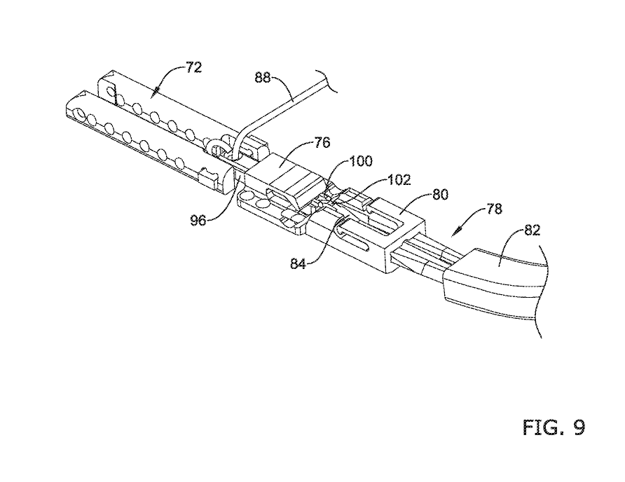

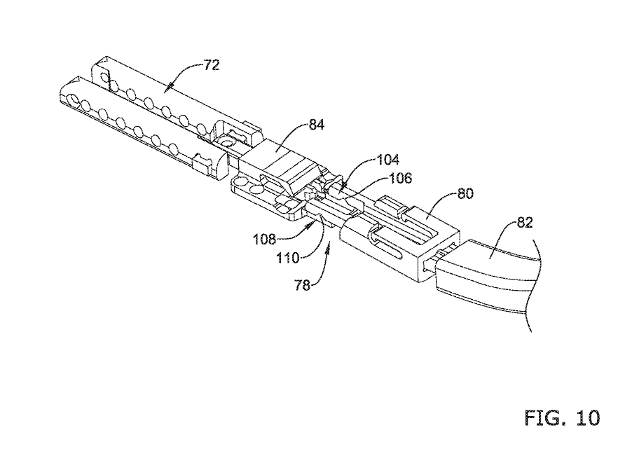

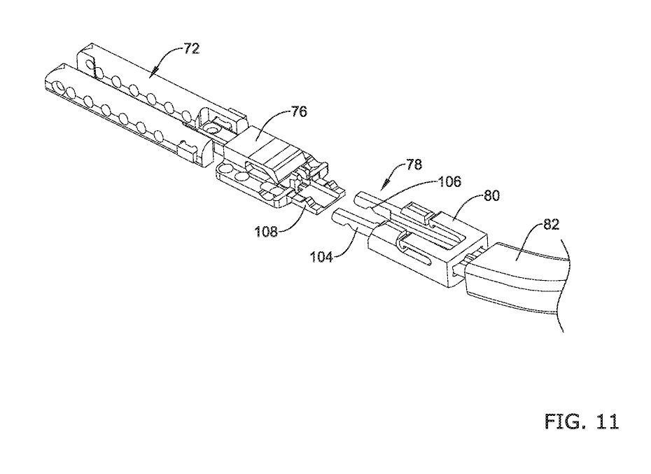

When the implant 16 reaches the intended target site within the anatomy, a clinician can proximally retract the push-pull rod 84, thereby moving the proximal ends of the posts 72 toward the distal ends of the buckles 76 in order to expand the implant 16. Ultimately, the push-pull rod 84 can be retracted sufficiently far enough to lock the post 72 with the buckle 76 so as to lock implant in an expanded configuration suitable for implantation within the anatomy. FIG. 9 illustrates the push-pull rod 84 proximally retracted. In doing so, the post 72 is brought into contact with the buckle 76. More particularly, a raised, generally transversely-oriented ridge 100 on the t-shaped bar portion 96 may be pulled proximally past the buckle 76 so that the post 72 is secured and held in place by the buckle 76. At this point, it is possible to urge the push-pull rods 84 distally to “unlock” the implant 16, thereby allowing for repositioning and/or retraction. Alternatively, if a clinician is satisfied with the positioning and/or locking of the implant 16 (e.g., after visualization of the implant 16 via a suitable imaging technique), the pins 88 may be pulled (e.g., removed from the openings 98 and the openings in the push-pull rods 84) to uncouple the push-pull rods 84 from the posts 72 as shown in FIG. 10. Further retraction of the push-pull rods 84 causes a longitudinally-oriented ridge 102 on the push-pull rods 84 to engage the collar 80 and causes the collar 80 to slide proximally along the fingers of the coupler 78. In doing so, a forked end 104 of the fingers, which has a groove 106 formed therein, is exposed and can be uncoupled from a rail 108, which has a projection 110 formed thereon that is configured to mate with the groove 106, as shown in FIG. 11. Thereafter, the system 10 can be removed from the anatomy, leaving behind the expanded and deployed the implant 16.

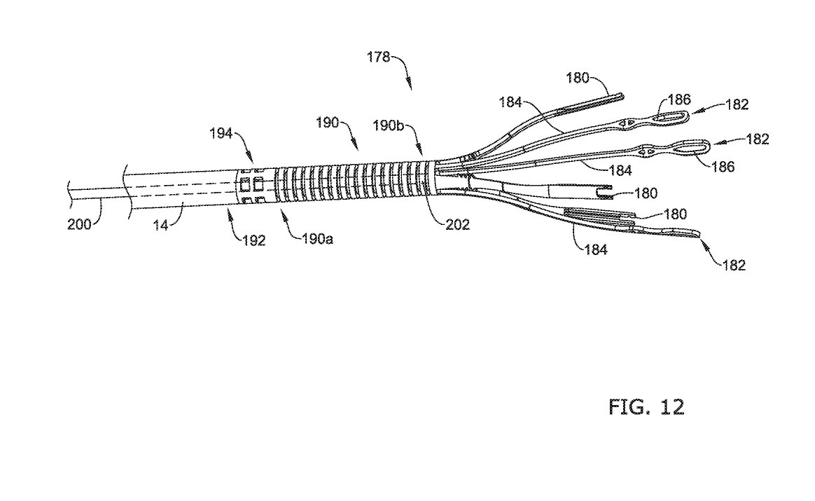

FIG. 12 shows the inner catheter 14 with an alternate coupler 178 previously discussed relative to FIG. 7 (e.g. 78). While the coupler 178 includes a total of three fingers 180 that are similar to the fingers shown in FIG. 7, the coupler 178 also includes a total of three sheathing aids 182. As discussed with respect to FIG. 7, each of the fingers 180 may be configured to engage with the implant 16 at posts 72 and buckles 76 (FIG. 7). Each of the sheathing aids 182 include a stalk 184 and a petal 186 arranged at the terminal end of each stalk 184. In some cases, for example, the sheathing aids 182 may be used to aid in sheathing the implant 16 into the outer sheath 12. In some cases, the sheathing aids 182 may also aid in the initial sheathing of the implant 16 (e.g. removing the implant 16 from a packaging container such as a bottle and pulling the implant 16 into the outer sheath 12) and in re-sheathing the implant 16 during repositioning and/or retraction of the implant 16 within the area of interest.

In some cases, the coupler 178 includes an elongate member 190 that extends between a distal end 192 of the inner catheter 14 to where the fingers 180 and the sheathing aids 182 start. The elongate member 190 may be considered as having a proximal region 190a and a distal region 190b. The elongate member 190 may, in some instances, include several windows 194 that are cut into the elongate member 190 for aiding in bonding the elongate member 190 to the inner catheter 14. In some cases, the coupler 178 may be laser cut from a single piece of metal, with the elongate member 190, each of the fingers 180 and each of the sheathing aids 182 all cut from that single piece of metal. In some cases, therefore, the coupler 178 may be considered as being integrally formed. Alternatively, in some cases, the fingers 180 and/or the sheathing aids 182 may instead be welded or soldered to the elongate member 190. In some cases, the flexibility of the elongate member 190 may aid in aligning the implant 16 with the native annulus, for example. In some cases, this may also reduce the forces necessary to forward load the implant 16 during deployment.

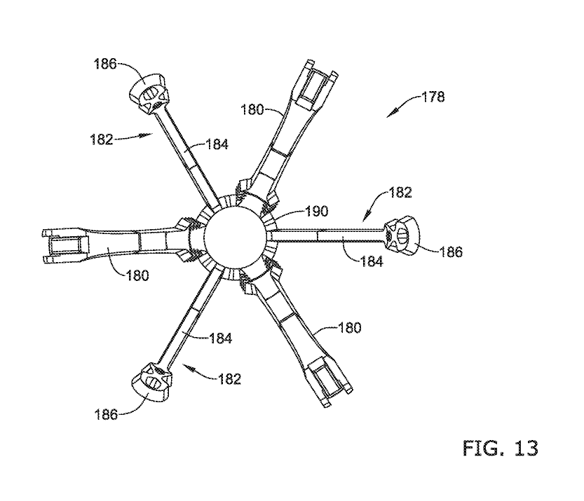

Because the relative spacing of the fingers 180 and the sheathing aids 182 is difficult to see in the perspective view of FIG. 12, FIG. 13 provides an end view of the coupler 178. As can be seen in FIG. 13, the three fingers 180 are each radially spaced about 120 degrees apart from one another. Similarly, the three sheathing aids 182 are each radially spaced about 120 degrees apart from one another, and each sheathing aid 182 is equally spaced between adjacent fingers 180. In some cases, then, there is a finger 180 or a sheathing aid 182 radially spaced about 60 degrees apart from a neighboring finger 180 or a neighboring sheathing aid 182. While a total of three sheathing aids 182 are illustrated, it will be appreciated that in some cases there may be more than three sheathing aids 182. While the three sheathing aids 182 are shown as being equally spaced apart, this is not required in all cases. In some cases, one or more of the sheathing aids 182 may vary in length. In use, in some cases, one or more of the sheathing aids 182 may be woven in and out of the braid 70 (FIG. 7) while one or more of the sheathing aids 182 may extend along an exterior of the braid 70, acting as more of a funnel for sheathing.

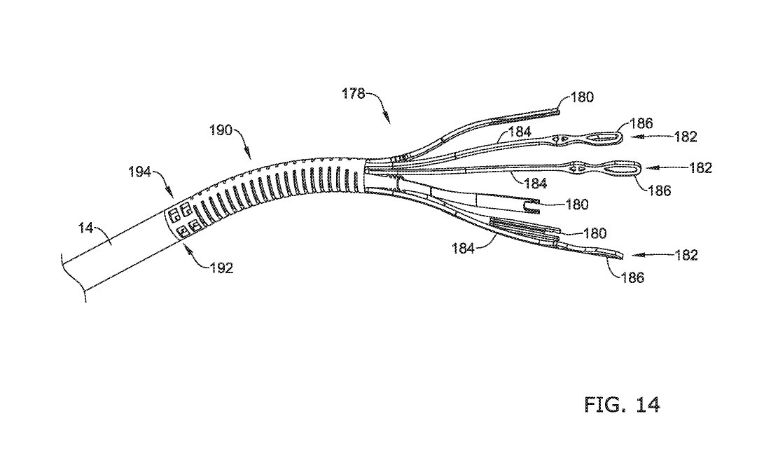

Returning to FIG. 12, in some cases an actuation member 200 may extend distally through the inner catheter 14 and the elongate member 190, as shown in phantom, and may terminate at a connection point 202 inside the elongate member 190. In some cases, the actuation member 200 may extend through one of the multiple lumens discussed as extending through the inner catheter 14, such as any of the lumens 46/48/50/52 discussed with respect to FIG. 5. In some cases, the actuation member 200 may extend proximally to the handle 18 and may be pushed or pulled, as desired, by engaging a linear or rotating actuator (not shown) added to the handle 18. Articulation may be achieved, for example, by pulling or shortening the actuation member 200. In some cases, by engaging the actuation member 200, the elongate member 190 may be deflected into a curved configuration, as shown for example in FIG. 14. For clarity, the actuation member 200 is not shown in FIG. 14.

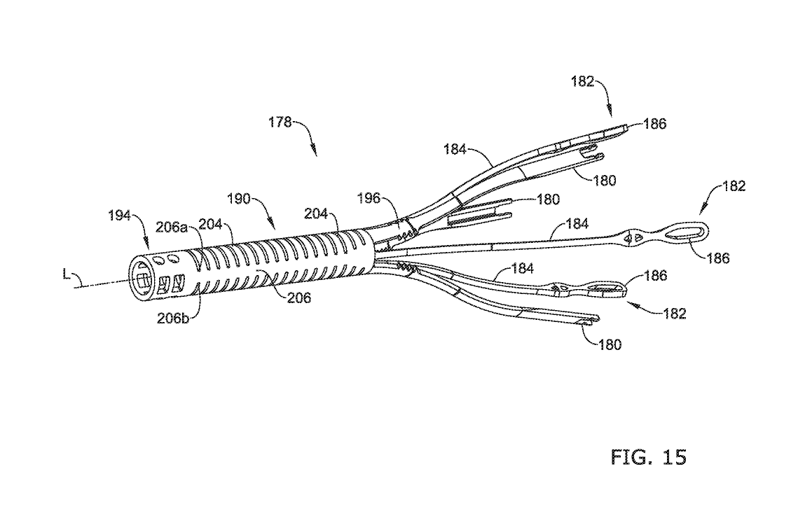

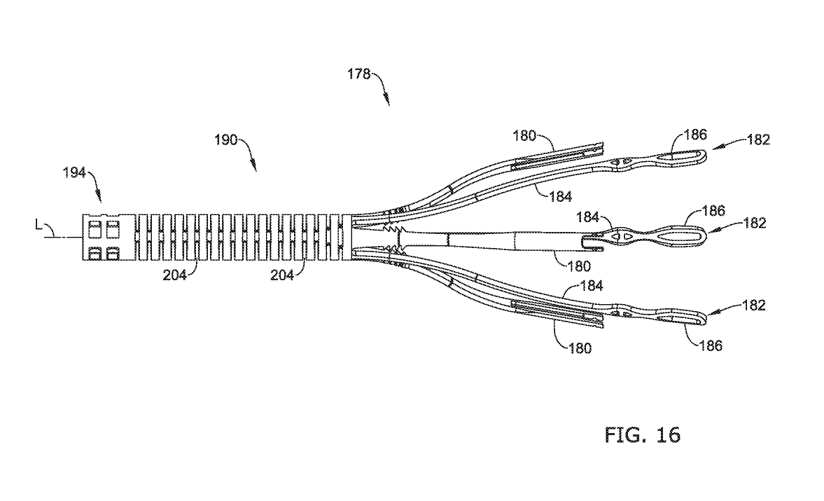

FIG. 15 is a perspective view of the coupler 178 and FIG. 16 is a perspective view showing the coupler 178 rotated about 180 degrees from the orientation shown in FIG. 15. As can be seen, the elongate member 190 includes a plurality of slots 204 that are cut into the elongate member 190. In some cases, the plurality of slots 204 are orientated orthogonal to a longitudinal axis L of the elongate member 190. In some instances, however, and depending on the desired bending behavior of the elongate member 190, at least some of the slots 204 may not be orthogonal to the longitudinal axis L of the elongate member 190. The slots 204 are cut a majority of the distance into the elongate member 190 such that the slots 204 form a spine 206, as shown in FIG. 15. In some cases, the slots 204 extend most of the way around the elongate member 190, such that each slot starts at a first side 206a of the spine 206 and extends all the way around to a second side 206b of the spine 206.

As illustrated, the elongate member 190 will have a preferential bending direction in which the slots 204 close, and the elongate member 190 bends away from the spine 206. To illustrate, in FIG. 15 the preferential bending direction may be considered as the petals 186 of the sheathing aids 182 bending into the plane of the paper, away from the viewer, while in FIG. 16 the preferential bending direction may be considered as the petals 186 of the sheathing aids 182 bending out of the plane of the paper, towards the viewer. It will be appreciated that the preferential bending direction, if there even is a preferential bending direction, may be controlled by how the slots 204 are cut into the elongate member 190 as well as where the actuation member 200 (FIG. 12) is attached to an interior of the elongate member 190.

In some cases, as shown, each of the fingers 180 may include a serrated region 196. In some cases, the serrated regions 196 may serve to help anchor a polymeric guide, such as the guides 82 referenced in FIG. 8. While the guides are not illustrated in FIG. 15 and FIG. 16, the guides, if present, may help to help keep each of the fingers 180 associated with a particular push-pull rod 84 such that the implant 16 may be manipulated.

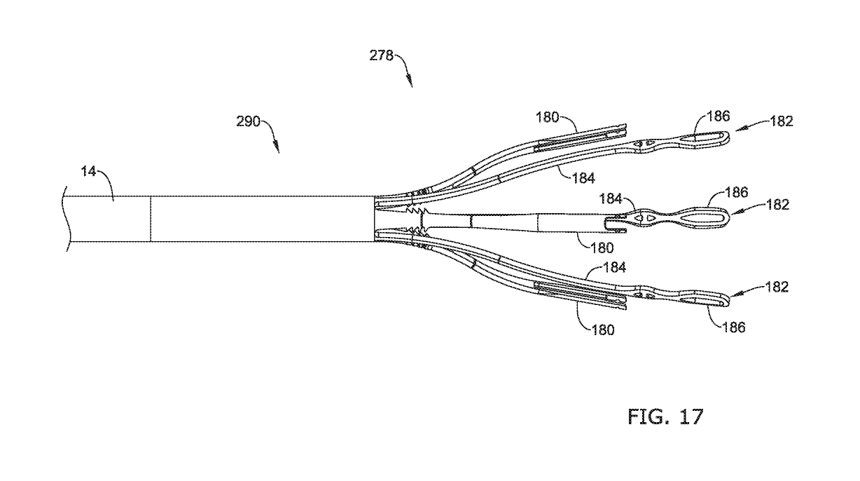

FIG. 17 is a perspective view showing a coupler 278 secured to the inner catheter 14. The coupler 278 includes an elongate member 290. In some instances, the elongate member 290 may be used in place of the elongate member 190 (FIG. 12), and in some cases, the elongate member 290 is a polymeric member formed of a relatively soft polymer. Examples of suitable polymers include PEBAX and polyurethane. In some cases, the elongate member 290 may be formed of a polymer having a durometer that is less than 60 D. In some cases, the elongate member 290 may be formed of a polymer having a durometer in the range of 25 D to 55 D. In cases in which the elongate member 290 is a polymeric member, the fingers 180 and the sheathing aids 182 may be separated formed, and may be secured, for example, via a metal band (not illustrated) that extends around the elongate member 290. In some cases, the elongate member 290 is a laser cut metal tube, much like the elongate member 190, with a polymeric liner or exterior polymeric coating.

The materials that can be used for the various components of the system 10 (and/or other systems disclosed herein) and the various tubular members disclosed herein may include those commonly associated with medical devices. For simplicity purposes, the following discussion makes reference to the outer sheath 12 and/or the inner catheter 14. However, this is not intended to limit the devices and methods described herein, as the discussion may be applied to other similar tubular members and/or components of tubular members or devices disclosed herein.

The outer sheath 12 and/or the inner catheter 14 may be made from a metal, metal alloy, polymer (some examples of which are disclosed below), a metal-polymer composite, ceramics, combinations thereof, and the like, or other suitable material. The coupler 178, 278 may be formed of a metal. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the outer sheath 12 and the inner catheter 14 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the outer sheath 12 and the inner catheter 14, or portions thereof, may be made of a material that does not substantially distort the image and create substantial artifacts (i.e., gaps in the image). Certain ferromagnetic materials, for example, may not be suitable because they may create artifacts in an MRI image. The outer sheath 12 and inner catheter 14, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the outer sheath 12 and the inner catheter 14 that may define a generally smooth outer surface for the system 10. In other embodiments, however, such a sheath or covering may be absent from a portion of all of the system 10, such that the outer sheath 12 and the inner catheter 14 may form an outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly praraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the system 10 (including, for example, the exterior surface of the outer sheath 12 and the inner catheter 14) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of the outer sheath 12 and the inner catheter 14, or other portions of the system 10. Alternatively, the sheath may comprise a lubricious, hydrophilic, protective, or other type of coating. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A medical device delivery system, comprising:
an outer shaft having an outer shaft lumen extending therethrough;
an inner shaft slidingly disposable within the outer shaft lumen, the inner shaft including a distal end region;
a coupler secured to the distal end region, the coupler comprising:
an elongate tubular member including a proximal region and a distal region, the proximal region of the elongate tubular member secured to the distal end region of the inner shaft;
a plurality of fingers extending distally from the distal region of the elongate tubular member, the plurality of fingers adapted to releasably engage an implantable medical device; and
a plurality of sheathing aids extending distally from the distal region of the elongate tubular member, the plurality of sheathing aids adapted to guide the medical device back into the outer shaft lumen.

2. The medical device delivery system of claim 1, wherein the elongate tubular member further comprises one or more windows cut into the proximal region of the elongate tubular member in order to facilitate securement of the elongate tubular member to the distal end region of the inner shaft.

3. The medical device delivery system of claim 1, wherein the coupler is laser cut from a single piece of metal.

4. The medical device delivery system of claim 3, wherein the plurality of fingers are part of the elongate tubular member.

5. The medical device delivery system of claim 3, wherein the plurality of sheathing aids are part of the elongate tubular member.

6. The medical device delivery system of claim 1, wherein at least some of the plurality of fingers and/or at least some of the plurality of sheathing aids are welded or soldered to the distal region of the elongate tubular member.

7. The medical device delivery system of claim 1, wherein the elongate tubular member is laser cut to provide the elongate tubular member with a spine running along one side of the elongate tubular member and a plurality of slots extending radially from the spine in order to provide the elongate tubular member with a preferential bending direction.

8. The medical device delivery system of claim 7, wherein the elongate tubular member preferentially bends in a preferential bending direction away from the spine, the plurality of slots closing up to enable the elongate tubular member to bend in the preferential bending direction.

9. The medical device delivery system of claim 1, wherein the coupler assembly further comprises a polymeric liner disposed about the elongate tubular member.

10. The medical device delivery system of claim 1, wherein at least some of the plurality of fingers include a serrated section adapted to secure a guide member to the finger.

11. The medical device delivery system of claim 1, wherein the elongate tubular member comprises a polymeric tubular member.

12. The medical device delivery system of claim 1, wherein the implantable medical device comprises an implantable heart valve.

13. A medical device delivery system, comprising:
an outer shaft having an outer shaft lumen extending therethrough;
an inner shaft slidingly disposable within the outer shaft lumen, the inner shaft including an inner shaft lumen extending therethrough, the inner shaft including a distal end region; and
a laser cut coupler secured to the distal end region, the laser cut coupler including an elongate tubular member and a plurality of fingers extending distally from the elongate tubular member, the plurality of fingers adapted to engage an implantable medical device;
wherein the elongate tubular member is laser cut to provide the elongate tubular member with a spine running along one side of the elongate tubular member and a plurality of slots extending radially from the spine in order to provide the elongate tubular member with a preferential bending direction in which the elongate tubular member bends away from the spine.

14. The medical device delivery system of claim 13, wherein the plurality of slots close up to enable the elongate tubular member to bend in the preferential bending direction.

15. The medical device delivery system of claim 13, further comprising a plurality of sheathing aids extending distally from the elongate tubular member, the plurality of sheathing aids adapted to guide the medical device back into the outer shaft lumen.

16. The medical device delivery system of claim 13, further comprising an actuation member extending through the inner shaft lumen and operably coupled to the elongate tubular member such that actuation of the actuation member causes the elongate tubular member to bend in the preferential bending direction.

17. The medical device delivery system of claim 13, wherein the elongate tubular member and the plurality of fingers are laser cut from a single piece of metal.

18. The medical device delivery system of claim 13, wherein the outer shaft has a curved distal end.

19. The medical device delivery system of claim 13, wherein the inner shaft has a curved distal end.

20. A medical device delivery system, comprising:
an outer shaft having an outer shaft lumen extending therethrough;
an inner shaft slidingly disposable within the outer shaft lumen;
a coupler extending distally from the inner shaft, the coupler comprising:
an elongate tubular member including a proximal region and a distal region, the proximal region of the elongate tubular member secured to the distal end region of the inner shaft;
a plurality of fingers extending distally from the distal region of the elongate tubular member, the plurality of fingers adapted to releasably engage an implantable heart valve; and
a plurality of sheathing aids extending distally from the distal region of the elongate tubular member, the plurality of sheathing aids adapted to guide the implantable heart valve back into the outer shaft lumen.
